(19) United States
(12) Patent Application Publication
(10) Pub. No.: US 2020/0108366 A1
(43) Pub. Date: Apr. 9, 2020

(54) APPARATUS FOR RETAINING SOLID MATERIAL IN A RADIAL FLOW REACTOR AND METHOD OF CONVERTING HYDROCARBONS

(71) Applicant: UOP LLC, Des Plaines, IL (US)

(72) Inventors: Michael J. Vetter, Schaumburg, IL (US); Sujay Krishnamurthy, Hoffman Estates, IL (US); Mohammad Reza Mostofi-Ashtiani, Naperville, IL (US)

(21) Appl. No.: 16/152,902

(22) Filed: Oct. 5, 2018

(51) Int. Cl. B01J 8/02; C07C 5/333

(52) U.S. Cl. CPC B01J 8/0242; B01J 2208/00849; C07C 5/333

(57) ABSTRACT

An apparatus for use in radial flow reactors is presented. The apparatus includes a first partition and a second partition with supports coupled therebetween. The first partition includes a first opening and a second opening to allow the passage of fluid therethrough. A baffle extends into a flow channel formed by adjacent support members to completely obstruct the first opening to interrupt a portion of the fluid flow therethrough. The baffle extends upwardly from one of the supports.

### FIELD OF THE INVENTION

This invention relates to cross or radial-flow reactors or adsorbers where a fluid flows across a bed of catalyst or adsorbent. In particular, this relates to the internal components of a reactor or adsorber for distribution flow of the fluid and for providing a device for preventing the flow of catalyst or adsorbent across the inlet or outlet screens.

### BACKGROUND OF THE INVENTION

A wide variety of processes use radial flow reactors to provide for contact between a fluid and a solid. The solid usually comprises a catalytic material on which the fluid reacts to form a product or the solid comprises an adsorbent material that retains one or more components in the fluid. The processes cover a range of processes, including hydrocarbon conversion, gas treatment, and adsorption for separation.

For example, radial flow reactors are constructed such that the reactor has an annular structure and that there are annular distribution and collection devices. The devices for distribution and collection typically incorporate some type of screened surface. The screened surface is for holding catalyst beds in place and for aiding in the distribution of pressure over the surface of the reactor to facilitate radial flow through the reactor bed. The screen can be a mesh, either wire or other material, or a punched plate. For either a fixed bed or moving bed, the screen or mesh provides a barrier to prevent the loss of solid catalyst particles while allowing fluid to flow through the bed. In a moving bed, solid catalyst particles are added at the top and flow through the apparatus and are removed at the bottom, while passing through a screened-in enclosure that permits the flow of fluid over the catalyst. In a fixed bed, the catalyst, or adsorbent, is loaded into a bed between screens, or other retention devices, and the screens allow fluid to flow over the catalyst while holding the catalyst in place. The screen is preferably constructed of a non-reactive material, but, in reality, the screen often undergoes some chemical reactions through corrosion and/or erosion, and over time problems arise from the corroded or eroded screen or mesh.

One type of screen is a profile wire screen, where a profile wire is wrapped around supports and set at a predetermined spacing for the wire as it is wrapped around the supports. The screen is then cut and flattened and then re-rolled or re-shaped. The screen is shown in U.S. Pat. Nos. 2,046,458 and 4,276,265. When re-rolled or re-shaped, the screen includes the profile wires, which are typically oriented vertically with support rods attached thereto and extending across the profile wires and orthogonally therefrom. The screen can be used as part of an inlet distribution device, or other device for containing a catalyst. One type of inlet distribution device is a reactor internal having a scallop shape and is described in U.S. Pat. No. 6,224,838 and U.S. Pat. No. 5,366,704. The scallop shape and design provides for good distribution of gas for the inlet of a radial flow reactor, but uses screens or meshes to prevent the passage of solids. The scallop shape is convenient because it allows for easy placement in a reactor without concern regarding the curvature of the vessel wall. The screens or meshes used to hold the catalyst particles within a bed are sized to have apertures sufficiently small that the particles cannot pass through.

In one common approach, profile wire screen(s) are formed into a generally tubular or cylindrical shape extending vertically within the generally vertical annular reactor about a central axis thereof. A perforated plate may be spaced from the profile wires and connected to opposite edges of the support rods on a fluid side of the screen within the reactor, while the profile wires are typically oriented on a material side. The plates are also formed or oriented to into a tubular or cylindrical shape within the reactor. Depending on the type of reactor and where within the reactor the screens are positioned, plates may be closer to the center or the outer walls of the reactor. As mentioned, the plates often include punched or perforated plates having a plurality of openings. Support rods may be oriented above and below the openings to provide a channel for fluid to flow from the openings in the plate to the openings or mesh in the profile wire screens to provide suitable distribution of the fluid to the solid catalyst or adsorbent bed. In one design, the reactor includes a centerpipe that includes an inner annular plate and an outer annular profile wire screen as described. Fluid flows from an inlet through the centerpipe and passes through the plate openings and out of the screen to contact the catalyst.

It has previously been identified that the fluid flowing through the plate openings and channels may cause jetting which, when contacting the screen and the solid material on the opposite side of the screen, causes vibration of the screen and/or solid material and accelerate corrosion or erosion of the outer surface of the profile wire screen and potentially damage the solid material. This can decrease the life of the equipment and catalyst or adsorbent within the reactor, increasing the cost of maintaining the reactor as well as down time required for changing out internal components of the reactor.

The design of reactors to overcome these limitations can save significantly on downtime for repairs and on the loss of catalyst, which is a significant portion of the cost of processing hydrocarbons. Accordingly, U.S. Pat. No 9,433,909, assigned to the present applicant, and the entirety of which is incorporated herein by reference, discloses an apparatus with a baffle extending downwardly to partially obstruct a flow path of the fluids as it moves through a first partition to the second partition. However, there remains an ongoing need for new designs for reactors to continually reduce the time for repair, as well as reduce the time to construct and maintain such reactors.

### SUMMARY OF THE INVENTION

An apparatus, and process of using an apparatus, according to the present invention mitigates screen erosion and jetting from fluids. Compared to current designs, the upstream perforated plate has an increased number of smaller diameter holes. This increased number of holes decreases the flow per hole to be conditioned. Additionally, the support has a baffle on the upper side of a support rod that is offset from the perforated plate at the leading edge of the support rod. The baffle extends up to cover the entire perforated plate hole opening. Thus, the baffle, along with the support, completely obstruct the opening in the perforated plate. The contracted flow into the openings of the perforated plate directly impinges on the baffle, is redirected both circumferentially and axially, expanded and decelerated. Thus, the dynamic energy of the stream is effectively removed, thereby reducing the erosive capability of the stream. Furthermore, it has been found, that an upwardly extending protrusion requires less accuracy in positioning compared to the downwardly extending protrusions, resulting in less time required for assembling and repairing the apparatus.

Therefore, the present invention may be characterized, in at least one aspect, as providing an apparatus for retaining a solid in a reactor in which the apparatus includes: a generally vertical fluid side partition having a fluid side opening to allow passage of the fluid therethrough; a generally vertical solid side partition spaced radially from the fluid side partition and generally parallel thereto having a solid side opening to allow passage of fluid therethrough; a fluid flow path between the fluid side partition and the solid side partition; and a baffle extending into the fluid flow path to completely obstruct the fluid side opening and interrupt the flow of fluid along the fluid flow path.

In one or more embodiments, the apparatus may further include a support extending between the fluid side partition and the solid side partition. The support may obstruct a portion of the fluid side opening. The baffle may be attached to the support. It is contemplated that the baffle is attached to an upper surface of the support. It is further contemplated that the baffle extends upwardly from the support to obstruct the fluid side opening.

In one or more embodiments, the baffle may be spaced from the generally vertical fluid side partition at a distance of 0.2 times a distance between the generally vertical fluid side partition and the generally vertical solid side partition.

In one or more embodiments, the fluid side opening may have a height of about 10 mm and the baffle may have a height 9 mm.

In one or more embodiments, the solid side partition may be a profile wire screen and the fluid side partition may be a perforated plate.

According to a second aspect, the present invention may also be broadly characterized as providing an apparatus for retaining a solid in a reactor, in which the apparatus includes: a profile wire screen including a plurality of generally vertically aligned and horizontally spaced profile wires; a plurality of supports each extending generally horizontally away from the profile wire screen and having a first end coupled to the profile wire screen; a plate spaced from the profile wire screen and coupled to second ends of the supports from the plurality of supports, the plate comprising a plurality of plate openings; a fluid flow path extending between the plate openings and the spaced profile wires; and, a baffle extending upwardly from an upper surface of each of the supports from the plurality of supports, the baffles obstructing the plate openings to interrupt a flow of fluid along the fluid flow path.

In one or more embodiments, the baffles may be spaced from the plate a distance of 0.2 times a distance between the plate and the profile wire screen.

In one or more embodiments, the plate openings are completely obstructed. The supports and the baffles may each partially obstruct the plate openings so that the plate openings are fully obstructed.

In one or more embodiments, the plate openings are fully obstructed, and the baffles have a height less than a height of the plate openings.

According to a third aspect, the present invention may also be generally characterized as providing a process for hydrocarbon conversion in a reactor that includes a catalyst, the catalyst retained by a support structure comprising a catalyst side partition including a plurality of generally vertically aligned and horizontally spaced profile wires, a plurality of supports each extending generally horizontally away from the catalyst side partition and each having a first end coupled to the catalyst side partition, a fluid side partition spaced from the catalyst side partition and coupled to second ends of each of the supports from the plurality of supports, the fluid side partition comprising a plurality of fluid openings, and, a flow path extending from the fluid openings to the horizontally spaced profile wires. The process includes: passing a hydrocarbon vapor into the reactor; flowing the hydrocarbon vapor through the fluid openings toward the catalyst side partition; redirecting the hydrocarbon vapor by completely obstructing the flow paths, wherein the flow paths are each interrupted by a baffle; and, flowing the hydrocarbon vapor through the spaced profile wires.

In one or more embodiments, flow paths may be also interrupted by the supports.

In one or more embodiments, each baffle may be coupled to one of the supports.

In one or more embodiments, the baffles may extend away from an upper surface of the supports.

In one or more embodiments, the baffles may be spaced from the fluid side partition at a distance of 0.2 times a second distance between the fluid side partition and the catalyst side partition.

In one or more embodiments, the baffles may have a height less than a height of the fluid openings.

Additional aspects, embodiments, and details of the invention, all of which may be combinable in any manner, are set forth in the following detailed description of the invention.

### DETAILED DESCRIPTION OF THE DRAWINGS

One or more exemplary embodiments of the present invention will be described below in conjunction with the following drawing figures, in which:

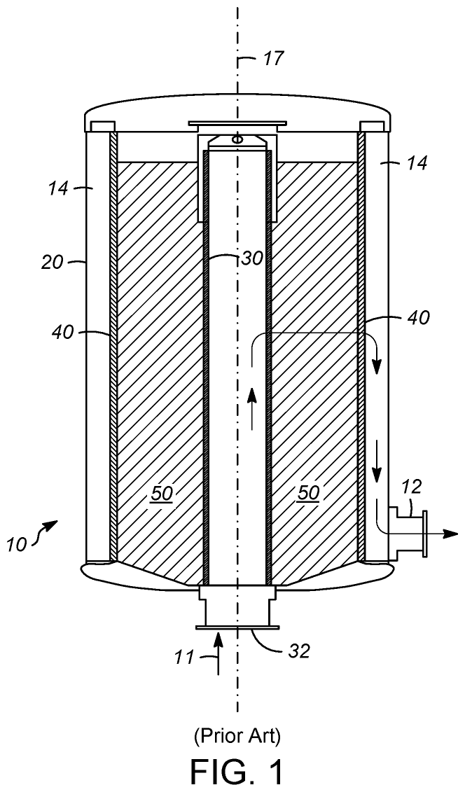

FIG. 1 shows a first reactor configuration that can be used in accordance with the present invention;

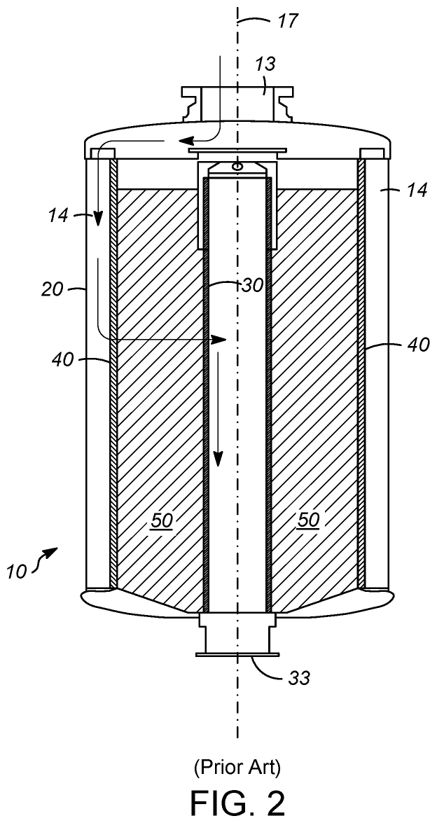

FIG. 2 shows a second reactor configuration that can be used in accordance with the present invention;

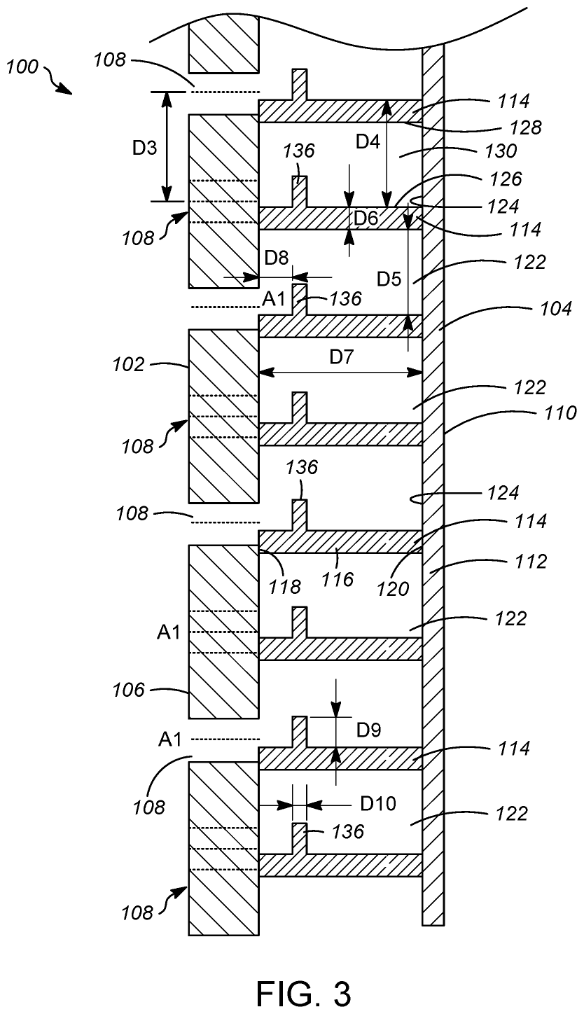

FIG. 3 shows a perspective, cutaway side view of an apparatus according to the present invention; and

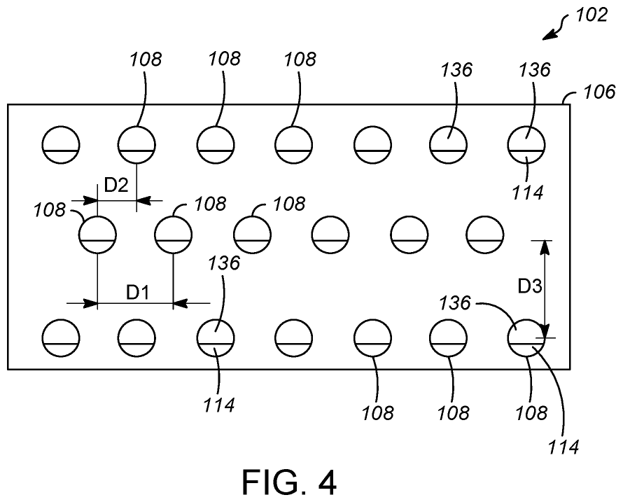

FIG. 4 shows a perspective, front view of the apparatus of FIG. 3.

### DETAILED DESCRIPTION OF THE INVENTION

As mentioned above, a new apparatus for use with a radial flow reactor, for example, has been invented. The apparatus includes baffles completely obstructing the flow path between the inner and outer partitions. The baffles extending upwardly from supports extending between the two partitions.

With these general principles in mind, one or more embodiments of the present invention will be described with the understanding that the following description is not intended to be limiting.

As radial flow reactors often produce harsh chemical environments and severe operating conditions in terms of pressure and temperature which creates tremendous stresses on the screens in these types of reactors. Thermal cycles and the weight of the catalyst can cause buckling of the screens. Stronger screens or devices for retaining catalyst are needed. Radial flow reactors, and cross-flow systems in general, need screens to contain the catalysts used in the reactors. Catalyst chamber internals grids are currently used in olefin production, for example, such as the dehydrogenation of propane to propylene or iso-butane to iso-butylene to support one or more adsorbent beds within the chamber or to separate individual adsorbent beds. The grids currently used are typically manufactured using profile wire construction.

Turning to FIG. 1, a radial flow reactor 10 in accordance with one aspect is illustrated that includes inner and outer annular partitions for supporting an annular bed of solid material therebetween. While the present description is provided in terms of a reactor system, the equipment and processes described herein are applicable to adsorbers, or other equipment used in contacting fluids with solids.

The reactor 10 includes a reactor shell 20, one partition in the form of a centerpipe 30, an outer partition in the form of screened partition 40, and a solid particle, or catalyst, bed 50. The reactor 10 by one aspect is configured so that fluid enters the reactor 10 through an inlet 32 at the bottom of the reactor and travels upwardly through the centerpipe 30 in the direction indicated by arrow 11. As the fluid flows upwardly, portions of the fluid are directed radially through the centerpipe, into the catalyst bed 50 where the fluid contacts the catalyst and reacts to form a product stream. The product stream flows radially outwardly through the outer screened partition 40 and into annular space 14 between the screened partition 40 and the reactor shell 20. The product stream is collected in the annular space 14 and passes through a reactor outlet 12.

According to another aspect illustrated in FIG. 2, the reactor may be configured to have an opposite flow pattern such that fluid enters through an inlet 13 and enters annular space 14 between the reactor shell 20 and the outer screened partition 40 and flows radially inwardly through the catalyst bed 50 where it contacts the catalyst and reacts to form a product stream. The product stream flows radially inwardly through the center pipe 30 where it is collected in the centerpipe and exits through the outlet 33.

Other configurations of the reactor 10 and flow are also possible and contemplated herein.

As currently practiced, where the reactor includes a radial outward flow configuration like that shown in FIG. 1, the centerpipe 30 includes an outer catalyst-side profile wire screen and an inner fluid-side perforated plate. The outer partition may also include an inner catalyst-side profile wire screen and/or an outer fluid-side perforated plate. Alternatively, where the reactor includes the radially inward flow configuration of FIG. 2, the outer partition 40 includes an inner catalyst-side profile wire screen and an outer fluid-side perforated plate. The centerpipe 30 may also include an outer catalyst-side profile wire screen and/or inner fluid-side perforated plate. In both of these configurations, the profile wire screen is subject to many stresses and a corrosive environment, including jetting of the fluid and vibration of one or both of the profile wire screen and the catalyst against the other, which can result in corrosion and erosion of the profile wire screen and damage to the catalyst.

The partitions 30 and 40 must perform the duty of preventing the passage of solid catalyst particles and allowing the passage of fluid, while providing structural strength to hold the catalyst against the pressure of the weight of the solid particles.

In accordance with one aspect, an apparatus for retaining a solid material in the reactor 10 is illustrated in FIGS. 3 and 4. The apparatus 100 includes a fluid-side partition 102 and a catalyst-side partition 104. As used herein, “fluid side” refers to the side or portion that is closer to the fluid within the reactor, such as closer to fluid flowing through centerpipe 30 or in the annular space 14, while “catalyst side” refers to a side or portion closer to the catalyst bed 50 or other solid material bed within the reactor. As described herein the apparatus may include or form a portion of the centerpipe 30 and/or the outer partition 40. For ease of explanation, the following will be described with regard to an apparatus for use as part of a centerpipe 30 within the outwardly radial flow configuration reactor of FIG. 1, although it should be understood that these principles and this description may be applied to the other reactor designs discussed above. Explanation of a reactor and components as having a cylindrical structure, is intended to include cylindrical structures, but also structures composed of individual planar components that when assembled make a multisided structure, such as having the cross sectional shape of an octagon or dodecagon, or any polygonal shaped cross-section, but can be substantially treated as a cylindrical structure.

By one aspect the fluid-side partition 102 includes a plate 106 having openings 108 therethrough. When positioned in the reactor 10 the plate 106 has an annular form about a center axis 17 of the reactor 10, and may be formed in different manners, including, for example, a single hollow cylindrical plate or tube or a plurality of flat or arcuate plates positioned circumferentially side-by-side about the axis 17. The openings 108 of the plate 106 extend through a thickness of the plate 106. The openings 108 may be circular or other shapes, including a variety of polygonal shapes or slots extending about the plate. As illustrated in FIG. 4, the plate includes round openings 108 that may be punched or drilled through the plate 106.

According to an aspect, the catalyst-side partition 104 includes a profile wire screen 110. U.S. Pat. Nos. 2,046,458 and 4,276,265, which are incorporated by reference herein, disclose typical structures and methods of making profile wire screens 110. The profile wire screen 110 includes a plurality of generally vertically oriented and horizontally spaced profile wires 112 and a plurality of generally horizontally oriented and vertically spaced supports 114 extending across the profile wires 112. The support members 114 extend generally orthogonally from the profile wires 112. It should be understood that for ease of description herein, terms such as “horizontal” and “vertical” are used to describe the partitions 102, 104 on a standalone basis as illustrated in the Figures. However, it should be understood that when the partitions 102, 104 are formed for being used in the reactor 10 or are positioned within the annular reactor 10, “horizontal” may refer to circumferential or radial, while “vertical” may refer to axial. For example, when situated within a reactor, the profile wires 112 will be generally axially oriented and circumferentially spaced about the center axis 17 of the reactor 10. Similarly, the orthogonally extending support members 114 may extend radially inward or outward depending on the configuration of the reactor 10.

The support members 114 include or comprise support rods 116. The support rods 116 may be coupled at an opposite edge portions 118, 120 between the plate 106 and the profile wires 112. The profile wires 112 and support rods 116 form openings or slots 124 through the profile wire screen 110 where they intersect.

The edge portions 118 of the supports 114 adjacent the plate 106 are vertically aligned above and below the openings 108 of the plate 106. In this manner an upper surface 126 of a support 114 and a lower surface 128 of an adjacent support 114 form a fluid channel 130 in fluid communication with the opening 108 and the slot 124. In this manner a plurality of fluid flow channels of fluid flow paths 122 are formed vertically along the apparatus 100 for the fluid to flow through the apparatus 100 or partitions 102, 104, for example centerpipe 30.

According to the present disclosure, a baffle 136 extends into the fluid flow path 122 to completely obstruct or interrupt a flow of fluid therethrough. By “completely obstructs” or “completely interrupts” it is meant that baffle 136, as well as other structures, such as support 114 (as shown in FIG. 4), completely obstruct the opening 108 when viewed along a center axis A1 of the opening 108. It has been identified that by positioning a baffle 136 within the flow path 122 in this manner, fluid jetting can be reduced as a result of flow redirection in multiple planes, circumferentially and axially, and a reduced, more even fluid velocity profile within the flow path 122 and along the profile wires 112 can be achieved to reduce localized peak velocities that cause vibration and erosion and corrosion of the profile wires screen 110 and/or the catalyst.

As shown in FIG. 3, the baffles 136 extend upwardly away from the upper surfaces 126 of the supports 114, providing, when viewed from the side (as in FIG. 3) an upside-down, or inverted, “T” shape. As opposed to having a baffle that extends downwardly, it has been found that the upwardly extending baffle 136 allows for a greater tolerance in the position of the baffle 136 between the plate 106 and the profile wires 112. This decreases the time associated with constructing and repairing the apparatus 100, since a less exact position is required to achieve the desired velocity reduction compared with prior designs.

According to one example, the openings 108 are circular and have a diameter of about 10 mm. By “about,” it is meant to include sizes ranging between 8 to 12 mm. Similar, ranges for “about” should be understood for the additional measurements by utilizing this ratio or range. For example, as shown in FIG. 4, the spacing D1 between adjacent openings 108 along a horizontal row is about 20 mm. The spacing D2 between an opening on a first horizontal row and an adjacent opening on an adjacent horizontal row is about 10 mm. As shown in FIGS. 3 and 4, the spacing D3 between adjacent horizontal rows of openings 108 is about 25 mm. Similarly, in FIG. 3 the spacing D4 between adjacent supports 114 is about 25 mm. The spacing D5 between an upper surface 126 of a support 114 and a lower surface 128 of an adjacent support 114 (positioned vertically above the first support) is about 20 mm. Thus, the support 114 preferably has a thickness D6 of about 5 mm. The distance D7 between the two partitions 102, 104 is about 38 mm. The baffle 136 is located a distance D8 away from the plate 106 of about 8 mm. The baffle 136 has a height D9 of about 7 mm, of which about 6.5 mm obstructs the flow path 122. The remaining 3.5 mm required to fully obstruct the flow path 122 is from the support 122. The baffle 136 has a thickness D10 of about 3 mm. The foregoing measurements are not intended to be limiting, but merely exemplary of the present invention.

When used, the present apparatus 100 is believed to reduce the erosion associated with fluid flowing into a device which includes the apparatus 100 by reducing the velocity of the fluid. Accordingly, in an exemplary process, a hydrocarbon vapor is passed into a reactor which includes the apparatus 100. The hydrocarbon vapor flows through the fluid openings 108 in the fluid side partition 102 toward the catalyst side partition 104. The hydrocarbon vapor is redirected in multiple planes, circumferentially and axially, as a result of the flow path 122 being completely obstructed. A least a portion of the complete obstruction is achieved with baffles 136 which interrupt the flow paths 122. The hydrocarbon vapor is then flowed through the spaced profile wires 112, for example, to be converted by the catalyst. The reduced velocity is less likely to erode the profiles wires, providing longer reactor life.

Any of the above lines, conduits, units, devices, vessels, surrounding environments, zones or similar may be equipped with one or more monitoring components including sensors, measurement devices, data capture devices or data transmission devices. Signals, process or status measurements, and data from monitoring components may be used to monitor conditions in, around, and on process equipment. Signals, measurements, and/or data generated or recorded by monitoring components may be collected, processed, and/or transmitted through one or more networks or connections that may be private or public, general or specific, direct or indirect, wired or wireless, encrypted or not encrypted, and/or combination(s) thereof; the specification is not intended to be limiting in this respect.

Signals, measurements, and/or data generated or recorded by monitoring components may be transmitted to one or more computing devices or systems. Computing devices or systems may include at least one processor and memory storing computer-readable instructions that, when executed by the at least one processor, cause the one or more computing devices to perform a process that may include one or more steps. For example, the one or more computing devices may be configured to receive, from one or more monitoring component, data related to at least one piece of equipment associated with the process. The one or more computing devices or systems may be configured to analyze the data. Based on analyzing the data, the one or more computing devices or systems may be configured to determine one or more recommended adjustments to one or more parameters of one or more processes described herein. The one or more computing devices or systems may be configured to transmit encrypted or unencrypted data that includes the one or more recommended adjustments to the one or more parameters of the one or more processes described herein.

It should be appreciated and understood by those of ordinary skill in the art that various other components such as valves, pumps, filters, coolers, etc. were not shown in the drawings as it is believed that the specifics of same are well within the knowledge of those of ordinary skill in the art and a description of same is not necessary for practicing or understanding the embodiments of the present invention.

### Specific Embodiments

While the following is described in conjunction with specific embodiments, it will be understood that this description is intended to illustrate and not limit the scope of the preceding description and the appended claims.

A first embodiment of the invention is an apparatus for retaining a solid in a reactor, the apparatus comprising a generally vertical fluid side partition having a fluid side opening to allow passage of the fluid therethrough; a generally vertical solid side partition spaced radially from the fluid side partition and generally parallel thereto having a solid side opening to allow passage of fluid therethrough; a fluid flow path between the fluid side partition and the solid side partition; and a baffle extending into the fluid flow path to completely obstruct the fluid side opening and interrupt the flow of fluid along the fluid flow path. An embodiment of the invention is one, any or all of prior embodiments in this paragraph up through the first embodiment in this paragraph, further comprising a support extending between the fluid side partition and the solid side partition. An embodiment of the invention is one, any or all of prior embodiments in this paragraph up through the first embodiment in this paragraph, wherein the support obstructs a portion of the fluid side opening. An embodiment of the invention is one, any or all of prior embodiments in this paragraph up through the first embodiment in this paragraph, wherein the baffle is attached to the support. An embodiment of the invention is one, any or all of prior embodiments in this paragraph up through the first embodiment in this paragraph, wherein the baffle is attached to an upper surface of the support. An embodiment of the invention is one, any or all of prior embodiments in this paragraph up through the first embodiment in this paragraph, wherein the baffle extends upwardly from the support to obstruct the fluid side opening. An embodiment of the invention is one, any or all of prior embodiments in this paragraph up through the first embodiment in this paragraph, wherein the baffle is spaced from the generally vertical fluid side partition at a distance of 0.2 times a distance between the generally vertical fluid side partition and the generally vertical solid side partition. An embodiment of the invention is one, any or all of prior embodiments in this paragraph up through the first embodiment in this paragraph, wherein the fluid side opening has a height of about 10 mm and the baffle has a height 9 mm. An embodiment of the invention is one, any or all of prior embodiments in this paragraph up through the first embodiment in this paragraph, wherein the solid side partition comprises a profile wire screen and the fluid side partition comprises a perforated plate.

A second embodiment of the invention is an apparatus for retaining a solid in a reactor, the apparatus comprising a profile wire screen including a plurality of generally vertically aligned and horizontally spaced profile wires; a plurality of supports each extending generally horizontally away from the profile wire screen and having a first end coupled to the profile wire screen; a plate spaced from the profile wire screen and coupled to second ends of the supports from the plurality of supports, the plate comprising a plurality of plate openings; a fluid flow path extending between the plate openings and the spaced profile wires; and, a baffle extending upwardly from an upper surface of each of the supports from the plurality of supports, the baffles obstructing the plate openings to interrupt a flow of fluid along the fluid flow path. An embodiment of the invention is one, any or all of prior embodiments in this paragraph up through the second embodiment in this paragraph wherein the baffles are spaced from the plate a distance of 0.2 times a distance between the plate and the profile wire screen. An embodiment of the invention is one, any or all of prior embodiments in this paragraph up through the second embodiment in this paragraph wherein the plate openings are completely obstructed. An embodiment of the invention is one, any or all of prior embodiments in this paragraph up through the second embodiment in this paragraph wherein the supports and the baffles each partially obstruct the plate openings so that the plate openings are fully obstructed. An embodiment of the invention is one, any or all of prior embodiments in this paragraph up through the second embodiment in this paragraph, wherein the plate openings are fully obstructed, and wherein the baffles have a height less than a height of the plate openings.

A third embodiment of the invention is a process for hydrocarbon conversion in a reactor that includes a catalyst, the catalyst retained by a support structure comprising a catalyst side partition including a plurality of generally vertically aligned and horizontally spaced profile wires, a plurality of supports each extending generally horizontally away from the catalyst side partition and each having a first end coupled to the catalyst side partition, a fluid side partition spaced from the catalyst side partition and coupled to second ends of each of the supports from the plurality of supports, the fluid side partition comprising a plurality of fluid openings, and, a flow path extending from the fluid openings to the horizontally spaced profile wires, the process comprising passing a hydrocarbon vapor into the reactor; flowing the hydrocarbon vapor through the fluid openings toward the catalyst side partition; redirecting the hydrocarbon vapor by completely obstructing the flow paths, wherein the flow paths are each interrupted by a baffle; and, flowing the hydrocarbon vapor through the spaced profile wires. An embodiment of the invention is one, any or all of prior embodiments in this paragraph up through the third embodiment in this paragraph, wherein the flow paths are also interrupted by the supports. An embodiment of the invention is one, any or all of prior embodiments in this paragraph up through the third embodiment in this paragraph, wherein each baffle is coupled to one of the supports. An embodiment of the invention is one, any or all of prior embodiments in this paragraph up through the third embodiment in this paragraph, wherein the baffles extending away from an upper surface of the supports. An embodiment of the invention is one, any or all of prior embodiments in this paragraph up through the third embodiment in this paragraph, wherein the baffles are spaced from the fluid side partition at a distance of 0.2 times a second distance between the fluid side partition and the catalyst side partition. An embodiment of the invention is one, any or all of prior embodiments in this paragraph up through the third embodiment in this paragraph, wherein the baffles have a height less than a height of the fluid openings.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

1. An apparatus for retaining a solid in a reactor, the apparatus comprising:
a generally vertical fluid side partition having a fluid side opening to allow passage of the fluid therethrough;
a generally vertical solid side partition spaced radially from the fluid side partition and generally parallel thereto having a solid side opening to allow passage of fluid therethrough;
a fluid flow path between the fluid side partition and the solid side partition; and
a baffle extending into the fluid flow path to completely obstruct the fluid side opening and interrupt the flow of fluid along the fluid flow path.

2. The apparatus of claim 1, further comprising a support extending between the fluid side partition and the solid side partition.

3. The apparatus of claim 2, wherein the support obstructs a portion of the fluid side opening.

4. The apparatus of claim 2, wherein the baffle is attached to the support.

5. The apparatus of claim 4, wherein the baffle is attached to an upper surface of the support.

6. The apparatus of claim 4, wherein the baffle extends upwardly from the support to obstruct the fluid side opening.

7. The apparatus of claim 1, wherein the baffle is spaced from the generally vertical fluid side partition at a distance of 0.2 times a distance between the generally vertical fluid side partition and the generally vertical solid side partition.

8. The apparatus of claim 1, wherein the fluid side opening has a height of about 10 mm and the baffle has a height 9 mm.

9. The apparatus of claim 1, wherein the solid side partition comprises a profile wire screen and the fluid side partition comprises a perforated plate.

10. An apparatus for retaining a solid in a reactor, the apparatus comprising:
a profile wire screen including a plurality of generally vertically aligned and horizontally spaced profile wires;
a plurality of supports each extending generally horizontally away from the profile wire screen and having a first end coupled to the profile wire screen;
a plate spaced from the profile wire screen and coupled to second ends of the supports from the plurality of supports, the plate comprising a plurality of plate openings;
a fluid flow path extending between the plate openings and the spaced profile wires; and,
a baffle extending upwardly from an upper surface of each of the supports from the plurality of supports, the baffles obstructing the plate openings to interrupt a flow of fluid along the fluid flow path, wherein the plate openings are completely obstructed.

11. The apparatus of claim 10 wherein the baffles are spaced from the plate a distance of 0.2 times a distance between the plate and the profile wire screen.

12. (canceled)

13. The apparatus of claim 10 wherein the supports and the baffles each partially obstruct the plate openings so that the plate openings are fully obstructed.

14. The apparatus of claim 10, wherein the baffles have a height less than a height of the plate openings.

15. A process for hydrocarbon conversion in a reactor that includes a catalyst, the catalyst retained by a support structure comprising a catalyst side partition including a plurality of generally vertically aligned and horizontally spaced profile wires, a plurality of supports each extending generally horizontally away from the catalyst side partition and each having a first end coupled to the catalyst side partition, a fluid side partition spaced from the catalyst side partition and coupled to second ends of each of the supports from the plurality of supports, the fluid side partition comprising a plurality of fluid openings, and, a flow path extending from the fluid openings to the horizontally spaced profile wires, the process comprising:
passing a hydrocarbon vapor into the reactor;
flowing the hydrocarbon vapor through the fluid openings toward the catalyst side partition;
redirecting the hydrocarbon vapor by completely obstructing the flow paths, wherein the flow paths are each interrupted by a baffle; and,
flowing the hydrocarbon vapor through the spaced profile wires.

16. The process of claim 15, wherein the flow paths are also interrupted by the supports.

17. The process of claim 15, wherein each baffle is coupled to one of the supports.

18. The process of claim 17, wherein the baffles extending away from an upper surface of the supports.

19. The process of claim 15, wherein the baffles are spaced from the fluid side partition at a distance of 0.2 times a second distance between the fluid side partition and the catalyst side partition.

20. The process of claim 15, wherein the baffles have a height less than a height of the fluid openings.
